(19) United States
(12) Patent Application Publication
(10) Pub. No.: US 2010/0256515 A1
(43) Pub. Date: Oct. 7, 2010

(54) COMMUNICATION WITH AND CONSCIOUSNESS-ASSESSMENT OF ANESTHETIZED SURGERY PATIENTS

(76) Inventor: Marc J. Egeth, Philadelphia, PA (US)

Correspondence Address: MENDELSOHN, DRUCKER, & ASSOCIATES, P.C., 1500 JOHN F. KENNEDY BLVD., SUITE 405, PHILADELPHIA, PA, 19102, US

(21) Appl. No.: 12/752,739

(22) Filed: Apr. 1, 2010

Related U.S. Application Data
(60) Provisional application No. 61/211,791, filed on Apr. 3, 2009.

(51) Int. Cl. A61B 5/0402; A61B 5/0488; A61B 5/11; A61B 19/00

(52) U.S. Cl. 600/544; 600/546; 600/595; 128/898

(57) ABSTRACT

In one embodiment, prior to undergoing surgery, a patient is trained in the use of an anesthesia-awareness detection (AAD) system to produce an observable output by attempting to move a muscle. The AAD system includes an electromyographic (EMG) signal monitor. While undergoing surgery, the patient is anesthetized with a sedative and a paralytic. During surgery, if the surgical team wishes to asses the consciousness state or communicative ability of the patient, then the surgical team asks the patient to move the muscle. If the patient is unable to speak due to the paralytic but is aware and attempts to comply, then the attempt is detected by the AAD system, which provides a corresponding output to inform the surgical team, which may take appropriate action to ask additional questions of the patient and/or address the patient's anesthesia awareness.

This application claims the benefit of the filing date of U.S. Provisional Application No. 61/211,791 filed on Apr. 3, 2009, the teachings of which are incorporated herein by reference in their entirety.

### BACKGROUND OF THE INVENTION

1. Field of the Invention

The current invention relates to communication with and consciousness-assessment of anesthetized surgery patients, and more specifically but not exclusively, to the use of muscle-motion signals for such communication and consciousness assessment.

2. Description of the Related Art

Patients undergoing major surgical procedures are typically anesthetized. General anesthesia has three main purposes achieved using three corresponding types of drugs. These goals are analgesia, unconsciousness, and immobilization. An analgesic reduces or eliminates sensations of pain so that patients do not endure the physical pain of surgery (e.g., having their skin cut). This effect of reducing pain is also known as antinociception. A hypnotic sedative reduces or eliminates consciousness so that patients do not witness or otherwise sense the surgical procedure while undergoing it, which most people would find rather disturbing. An immobilizer (also called a paralytic) uses one or more of (i) a muscle relaxant, (ii) an autonomous-nervous-system suppressor, and (iii) a neuro-muscular-transmitter blocker, so that patients are immobilized and do not intentionally or unintentionally move during surgery and thereby injure themselves and/or members of the surgical team. Suppressing the autonomous nervous system also provides additional benefits, such as mitigating potentially harmful automatic responses of the body to the surgery.

Since each patient is unique, no single dosage of an anesthetic is likely to be appropriate for all patients. In addition, providing an under- or over-dosage of an anesthetic is highly undesirable. An under-dosage fails to achieve the desired anesthetic effect. An over-dosage wastes potentially expensive anesthetic, prolongs recovery, and can harm the patient. In order to ensure proper dispensation of anesthetics during surgery, a surgical team includes an anesthesiologist who administers the various anesthetics and monitors the patient's vital signs (e.g., pulse, blood pressure, heart activity, and brain activity) in order to determine and adjust dosages as necessary during the surgery. Nevertheless, under- and over-dosages of anesthetics do occasionally occur.

In a small fraction of surgeries, a patient regains consciousness in the middle of surgery while still anesthetized. This occurrence is known as anesthesia awareness or intraoperative awareness. In an episode of anesthesia awareness, the patient is conscious while being operated on but, because he or she is immobilized, the patient cannot communicate with the surgical team and alert it to this unfortunate circumstance. Regardless of whether or not the patient endures physical pain during such an episode (e.g., if the patient received insufficient analgesic), the patient may suffer psychological trauma from the experience of awaking helpless, immobile, exposed, cut open, and unable to communicate while continuing to be operated on and ignored by the surgical team. The trauma may cause nightmares and/or post-traumatic stress disorder. Even if the patient does not later consciously remember the experience, subconscious trauma may persist.

One method used to determine anesthesia awareness is known as Tunstall's isolated forearm technique (IFT). IFT involves tying a tourniquet around one arm of the patient before administering the muscle paralytic so that (i) the paralytic does not affect that arm and (ii) that arm remains free to move at the patient's command. Subsequently, during surgery, if the patient regains consciousness, then the patient can move that arm to alert the surgical team to the situation. However, using the IFT presents several problems. Use of the tourniquet for even a relatively short length of time can cause tissue damage. In addition, using IFT can interfere with regular surgical procedures, such as placement of intravenous feeds (IVs) in the arm. Furthermore, having a patient suddenly flail his or her arm in the middle of surgery may startle the surgical team and cause surgical mishaps and/or injuries. As a result, IFT is generally limited to use in research studies.

Other methods used to determine anesthesia awareness involve monitoring various vital signs of the patient that correlate with consciousness. Commonly, the signals monitored are electro-encephalogram (EEG) signals, which indicate brain activity. Various methods of analyzing the EEG signals are known, which involve processing the raw EEG data in an attempt to accurately assess a patient's consciousness level. As described, for example, in U.S. Pat. No. 7,367,949 to Korhonen et al. (“Korhonen”), EEG signal data is obtained from electrodes placed on a patient's scalp, where the EEG may be contaminated by electromyographic (EMG) signals which indicate muscular activity underneath the electrodes. The EEG signal data is filtered to remove the EMG signals (based on their different frequency bandwidth), since the EMG signals are considered artifacts with respect to the EEG signals. Korhonen then teaches using cardiovascular, EEG, EMG, and/or other signals to derive parameter values for use in a mathematical formula to calculate probability of patient comfort.

In another example, U.S. Pat. No. 6,801,803 to Viertio-Oja (“Viertio-Oja”) discloses combining an EEG-based spectral-entropy measurement with an EMG-based spectral-power measurement to automatically indicate hypnotic state or depth of anesthesia for a patient in surgery. Viertio-Oja teaches using the sudden appearance of EMG signal data as an indication of possible reaction to painful stimuli that, if unaddressed, can lead to eventual arousal from unconsciousness.

In yet another example, U.S. Pat. App. Pub. No. 2006/0217628 to Huiku (“Huiku”) teaches a device to automatically determine the anesthetic state of a patient by combining indices indicative of the patient's nociception (pain perception) and hypnosis levels. Huiku teaches determining the hypnosis level by evaluating the complexity or disorder (entropy) of the EEG signal.

### SUMMARY OF THE INVENTION

One embodiment of the invention can be an anesthesia-awareness detection (AAD) system comprising an output module, a muscle-motion sensor, and a signal processor. The output module is adapted to be activated to generate at least one of an audible output and a visible output. The muscle-motion sensor is adapted to (i) detect one or more muscle-motion signals produced by an anesthetized patient and (ii) generate a sensor output signal corresponding to the one or more muscle-motion signals. The signal processor is adapted to (i) process the sensor output signal to determine whether or not the one or more muscle-motion signals are volitional by the patient and (ii) activate the output module if the signal processor determines that the one or more muscle-motion signals are volitional indicating that the patient is aware.

Another embodiment of the invention can be a method for detecting anesthesia awareness comprising (a) detecting one or more muscle-motion signals produced by an anesthetized patient, (b) generating a sensor output signal corresponding to the one or more muscle-motion signals, (c) processing the sensor output signal to determine whether or not the one or more muscle-motion signals are volitional by the patient is aware, and (d) activating an output module if the signal processor determines that the one ore more muscle-motion signals are volitional indicating that the patient is aware, wherein the output module generates at least one of an audible output and a visible output if activated.

Yet another embodiment of the invention can be a method comprising (a) anesthetizing a patient, (b) then determining that the anesthetized patient may be aware, (c) then asking the anesthetized patient to perform an activity that would generate a detectable muscle-motion signal, (d) then observing an output module of an anesthesia-awareness system to determine whether a corresponding muscle-motion signal is detected, (e) then determining (1) that the patient is aware if the corresponding muscle-motion signal is detected and (2) the patient is probably not aware if the corresponding muscle-motion signal is not detected, and (f) then administering additional sedative to the patient if it was determined that the patient is aware.

### BRIEF DESCRIPTION OF THE DRAWINGS

Other aspects, features, and advantages of the present invention will become more fully apparent from the following detailed description, the appended claims, and the accompanying drawings in which like reference numerals identify similar or identical elements.

FIG. 1 shows a simplified block diagram of an anesthesia-awareness detection (AAD) system in accordance with one embodiment of the present invention.

FIG. 2 shows a flow chart for the exemplary utilization of the AAD system of FIG. 1 in accordance with one embodiment of the present invention.

### DETAILED DESCRIPTION

One of the more potentially traumatizing aspects of anesthesia awareness, aside from the unexpected and disturbing startling awareness of undergoing surgery, is the patient's unexpected inability to communicate with members of the surgical team operating on the patient. Providing systems and methods to allow an anesthetized, but aware patient to communicate with the surgical team may significantly reduce the trauma of such an event. Communicating with the surgical team by, for example, answering questions through manipulation of his or her own mental state in ways that are neurologically detectable, allows an anesthetized, but aware patient to thereby demonstrate awareness and also provide meaningful information to the surgical team.

During surgery, surface electrodes are typically placed on the patient's forehead in order to detect brain waves in the form of EEG signals. These electrodes also typically pick up muscular activity underneath the electrodes, detected in the form of EMG signals, which, as noted above, are ordinarily considered artifacts contaminating the desired EEG signals. It should be noted that, when EMG signals are the desired data, they are sometimes obtained using an invasive procedure involving the insertion of probe electrodes into muscle tissue. Note that, as used herein, the term “electrode” may refer to either a surface electrode or a probe electrode. Note also, that, as used herein, the term “EMG signals” refers to the electrical and/or action-potential signals produced by the activity of neurologically stimulated muscle cells. Note that EMG signals may be generated and detected as a result of volitional muscle activations even in the absence of any detectable muscle movement. Note further that muscular activity in a particular muscle is typically precipitated by the release of an appropriate neurotransmitter by the corresponding motor neuron, which, in turn, is caused by the propagation of an appropriate neurological signal along the motor neuron and zero or more different connected neurons. The propagation of the neurological signal, the release of the neurotransmitter, and other precursors to the EMG-generating muscle activity, which are also detectable to various extents by various means, are referred to herein as “neuromuscular signals.”

Depending on the particular type, dosage, and administration of a muscle paralytic, EMG-signal-producing activity of particular muscle locations may be drastically curtailed. Similarly, neuromuscular signals in particular locations may be drastically affected by particular types, dosages, and administrations of anesthetic drugs. Consequently, multiple situation-appropriate strategies are presented herein to allow the surgical team to detect an anesthesia-awareness event and to communicate with an anesthetized but aware patient.

In one typical situation noted above, surface electrodes are placed on a patient's forehead to obtain EEG data. If the patient attempts to furrow his or her brow, then corresponding EMG signals should be generated and detected. It should be noted that anesthetics may be administered such that the patient may retain the ability to fully or partially furrow his or her brow while otherwise substantially immobilized, since the miniscule movement involved in brow-furrowing is not likely to endanger the patient or the surgical team. Note that, as used here, the term “substantially immobilized” describes patients that are immobilized such that they cannot substantially move their limbs and related major muscles. Substantial immobilization may be achieved wholly with anesthetics or partially with anesthetics and partially with physical restraints. Patients who are substantially immobilized but not completely immobilized may be able to move relatively minor muscles, such as some facial muscles. Completely immobilized patients are completely paralyzed and unable to voluntarily move any muscles. Note, however, that even a completely immobilized patient may be able to generate an EMG signal in a muscle by trying to move that muscle.

The furrowing of the brow should generate detectable EMG signals that can be used to determine awareness and/or allow communication from the patient to the surgical team. Note that the mere furrowing of the patient's brow in the absence of a device monitoring for such furrowing is likely to go unnoticed by the surgical team during an operation since (1) the surgical team is likely to be focusing on the surgery and not be monitoring the patient's brow and (2) the patient may be completely immobilized and have no visible movement.

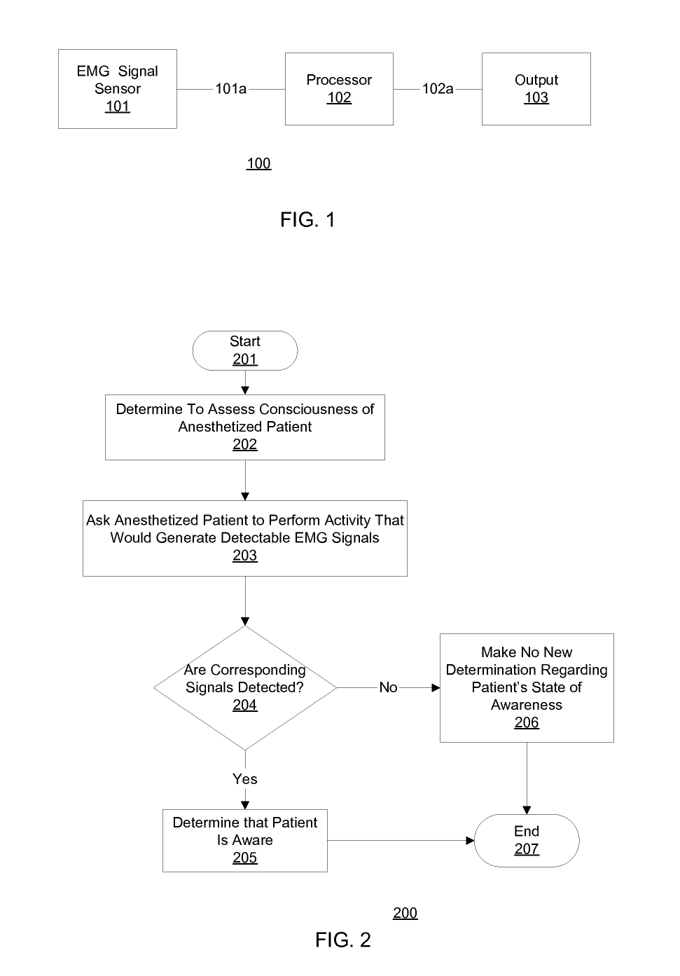

FIG. 1 shows a simplified block diagram of anesthesia-awareness detection (AAD) system 100 in accordance with one embodiment of the present invention. AAD system 100 comprises EMG (EMG) signal sensor 101, processor 102, and output module 103. Sensor 101 is connected to processor 102 via path 101a. Processor 102 is connected to output module 103 via path 102a. Sensor 101 comprises an array of one or more electrodes for sensing the target signal set.

In one implementation of AAD system 100, sensor 101 comprises nine surface electrodes arranged in a 3-by-3 grid, where the surface electrodes are used to detect both EEG and EMG signals. A multiplicity of electrodes allows for more-advanced signal processing having a better signal-to-noise ratio (SNR) than with fewer electrodes. Note that, in alternative implementations, multiple, n*m electrodes may be arranged in any n-by-m grid, where m and n are integers and m>1. Sensor 101 may include analog electrical components, such as, for example, resistors, capacitors, and diodes, to condition the raw electrode signals before transmission to processor 102. Sensor 101 provides sensor output signal 101a to processor 102. Sensor output signal 101a corresponds to the electrode signals by, for example, indicating signal amplitudes or power levels over time.

Processor 102 processes the sensor output signal to determine whether the signal indicates sensed motion or attempted motion. In one implementation, processor 102 calculates a metric based on the received signal data, and, if the metric exceeds a set threshold, then processor 102 determines that motion occurred or was attempted, while, if the threshold is not exceeded, then processor 102 determines that motion neither occurred nor was attempted. The metric may be based on average signal amplitude over a defined period of time. The threshold may be pre-programmed or may be set during pre-operative preparation, as described elsewhere herein. Processor 102 then indicates the result of that determination through control of the output of output module 103. In one implementation, output module 103 comprises a visible light-emitting diode (LED) that lights up if processor 102 determines that motion occurred or was attempted.

FIG. 2 shows flowchart 200 for the exemplary utilization of AAD system 100 of FIG. 1 in accordance with one embodiment of the present invention. The process starts (step 201) with a determination to assess the consciousness of an anesthetized patient (step 202). The determination may be triggered by, for example, the output of output module 103, by the output of an automated patient-consciousness-monitoring system (not shown), or at the discretion of the surgical team. For example, an anesthetized patient who becomes aware may attempt to furrow his or her brow, consequently causing the LED output module 103 to light up and causing a determination of potential awareness. In another example, an automated consciousness-monitoring system may determine and indicate that the patient is actually or likely-to-soon-become aware. In yet another example, the surgical team may choose, for whatever reason, to determine that the patient is potentially aware.

After determining potential awareness (step 202), the anesthetized patient is asked to perform an activity that would generate detectable EMG signals (step 203). For example, the anesthesiologist may tell the patient, “If you can hear me, please try to furrow your brow.” If the patient is aware and responsive, then the patient can volitionally attempt to furrow his or her brow. It is then determined whether corresponding signals are detected (step 204) by, for example, AAD system 100. If corresponding signals are detected in step 204, then it is determined that the patient is aware (step 205), the procedure terminates (step 207), and appropriate follow-up action is undertaken (not shown), such as, for example, explaining the situation to the patient, providing comforting reassurance, and administering additional anesthetics to return the patient to unconsciousness. If corresponding signals are not detected in step 204, then no new determination is made regarding the patient's state of awareness (step 206), and the procedure terminates (step 207). Note that, although step 206 makes no new determination regarding the patient's state of awareness, the patient is most likely not aware, and the surgical team will likely continue with the surgery.

Using an automated consciousness-monitoring system, such as one of the above-described systems, in combination with an embodiment of the present invention may provide additional assurance that a patient undergoing surgery is not aware while anesthetized. If using such a combination, then the surgical team may adjust the sensitivity of the automatic monitoring system to make it more sensitive to possible awareness and, thereby, reduce the incidence of false negatives, where a patient becomes aware without triggering the procedure described in FIG. 2. If an automatic monitor is used alone, increasing its sensitivity would likely cause too many false positives, where the monitor incorrectly determines that the anesthetized patient is aware. However, in combination with the embodiment of the present invention, those positives are merely triggers for further processing during which false positives are discarded while false negatives are made less likely.

In one embodiment, AAD system 100 of FIG. 1 is implemented as a unitary mobile device containing a power source (e.g., a rechargeable battery) and the above-described components. AAD system 100 would be placed on the patient's forehead. Subsequently, during surgery, if the LED of output module 103 lights up, then the surgical team would be alerted to the fact that the patient may be aware. The patient would then be able to communicate with the surgical team by consciously manipulating his brow and, thereby, activate output module 103. The communication could be as simple as outlined by flowchart 200 of FIG. 2, or could comprise more-complex communication, such as responding to commands, answering questions, or volunteering some other information of the patient's choosing.

In one embodiment, AAD system 100 of FIG. 1 is programmable to allow for more-complex communication by the patient with the surgical team. The programming might require a particular pattern of acts by the patient, which are detectable by processor 102, in order to have processor 102 trigger a corresponding visible output from output module 103. The programming might vary the output of output module 103 depending on the detected pattern so that each particular detected pattern corresponds to a different output of output module 103. For example, AAD system 100 might indicate two furrows as a “no” and three as a “yes,” where “yes” and “no” may be indicated by differently colored LEDs of output module 103. Enabling an anesthetized but aware patient to respond to questions with a “yes” or “no” allows the surgical team to better assess and handle an occurrence of anesthesia awareness. For example, the surgical team might ask the anesthetized but aware patient whether he or she is feeling any pain. AAD system 100 may be programmed to also account for the temporal lengths of furrows and/or pauses. For example, in an implementation where output module 103 comprises an alphanumeric visual display, processor 102 may be able to interpret lengthy and brief furrows by the patient and intermediary pauses as Morse code and cause the corresponding alphanumeric characters to be displayed by output module 103.

AAD system 100 may be tested and/or calibrated for a particular patient during a pre-operative preparation procedure. The testing and calibration allows the patient to practice manipulating output module 103 and allows the patient and surgical team to (i) make adjustments necessary to allow such manipulation and (ii) confirm the patient's ability to do so when desired. The calibration may be used to adjust for the patient's idiosyncratic EMG levels during stillness and during actual movement in order, for example, to establish baseline and expected EMG levels for stillness and for movement. The surgical team may explain the operation and/or purpose of AAD system 100 and may instruct the patient on particular codes to use to communicate with the team, if needed. Alternatively, the surgical team might reserve such explanations for only the rare situations where an anesthesia-awareness event is suspected or known to be occurring.

An embodiment of the invention has been described where AAD system 100 of FIG. 1 is a unitary mobile device. In one alternative embodiment, AAD system 100 comprises two or more separate components that communicate using wired and/or wireless transmission. In a wired implementation, at least one of paths 101a and 102a is in the form of a flexible conductive cable. In a wireless implementation of AAD system 100, at least one of paths 101a and 102a represents a wireless transmission path. In one alternative embodiment, AAD system 100 is connected to a relatively fixed power source and, consequently, has limited mobility.

Embodiments of the invention have been described where output module 103 of FIG. 1 provides a visible output produced by lights and/or a visual display. In alternative embodiments, output module 103 also provides an audible output, such as beeps, pre-programmed sounds, pre-programmed voice alerts, or input-dependent synthesized sounds and/or speech. In alternative embodiments, output module 103 provides only an audible output without any visible output.

Embodiments of the invention have been described where sensor 101 of FIG. 1 senses EMG signals. In one alternative embodiment of AAD system 100 of FIG. 1, sensor 101 comprises an NM-signal detector for detecting NM signals associated with a particular muscle. Sensor 101 is adapted to (a) be placed proximate to a motor neuron associated with the muscle, (b) detect a neuromuscular (NM) signal generated by at least one of (i) the release of neurotransmitters by the motor neuron and (ii) the transmission of an impulse along the motor neuron, and (c) generate sensor output signal 101a indicative of the detected NM signal. In another alternative embodiment, sensor 101 comprises an NM-signal detector but no EMG-signal detector.

As noted elsewhere herein, in some surgeries, the patient may be able to move facial muscles while otherwise substantially immobilized. In such situations, it may be beneficial to use motion-sensing sensors, such as accelerometers, on facial muscles. In one alternative embodiment, sensor 101 also comprises an array of one or more surface-mounted accelerometers that (a) are adapted to be placed over a muscle, (b) can sense physical movements of the muscle, and (c) are adapted to generate sensor output signal 101a indicative of the movements of the muscle. In another alternative embodiment, sensor 101 comprises accelerometers and no EMG or neuromuscular-signal sensors. In yet another alternative embodiment, sensor 101 comprises accelerometers and neuromuscular-signal sensors but no EMG sensors. Collectively, (a) EMG signals, neuromuscular signals, and muscle-motion-activated accelerometer signals are referred to herein as muscle-motion signals and (b) sensors for detecting muscle-motion signals (e.g., EMG signals, neuromuscular signals, and muscle-motion-activated accelerometer signals) are referred to herein as muscle-motion sensors.

Embodiments of the invention have been described where sensor 101 of FIG. 1 comprises one or more surface electrodes placed on the patient's forehead. In alternative embodiments, one or more probe electrodes are used instead of one or more surface electrodes. In other alternative embodiments, electrodes are placed over body areas other than the forehead. Placing electrodes over other body parts, such as limbs, may be particularly useful in any of the following situations: (a) the electrode detects non-EMG neuromuscular activity, (b) the immobilizer used on the limbs does not significantly reduce EMG activity, or (c) the limbs supporting electrodes are immobilized with mechanical restraints rather than anesthetic ones (in other words, any corresponding muscle activity would be isometric). It should be noted that, in embodiments where sensor 101 is placed on locations other than the head, sensor 101 would not likely be able to detect EEG signals. Since EEG signals are typically measured during surgery, the patient will likely still have a set of electrodes on his or her forehead. The signals from the forehead electrodes may be used in conjunction with the signals from sensor 101 and processed by processor 102 or those forehead-electrodes signals may be ignored by AAD system 100.

Embodiments of the invention have been described where sensor 101 of FIG. 1 is placed on substantially one location on the body. In alternative embodiments, the electrodes of sensor 101 are placed on substantially different locations on the patient. In one such alternative embodiment, some electrodes are placed on the patient's right arm, and some electrodes are placed on the patient's left arm. This disparate placement may be used to encode communication from the patient, such as using an attempt to move one arm to signify “yes” and using an attempt to move the other arm to signify “no.”

While allowing a surgical team to determine that an anesthetized patient undergoing surgery is nevertheless conscious and aware, some embodiments of the invention described above may provide additional benefits, such as allowing the patient to communicate with the surgical team and avoiding complex mathematical computation and analysis of signal data.

As used herein in reference to data transfers between entities in the same device, and unless otherwise specified, the terms “receive” and its variants can refer to receipt of the actual data, or the receipt of one or more pointers to the actual data, wherein the receiving entity can access the actual data using the one or more pointers.

Exemplary embodiments have been described wherein particular entities (a.k.a. modules) perform particular functions. However, the particular functions may be performed by any suitable entity and are not restricted to being performed by the particular entities named in the exemplary embodiments.

Exemplary embodiments have been described with data flows between entities in particular directions. Such data flows do not preclude data flows in the reverse direction on the same path or on alternative paths that have not been shown or described. Paths that have been drawn as bidirectional do not have to be used to pass data in both directions.

The present invention may be implemented as circuit-based processes, including possible implementation as a single integrated circuit (such as an ASIC or an FPGA), a multi-chip module, a single card, or a multi-card circuit pack. As would be apparent to one skilled in the art, various functions of circuit elements may also be implemented as processing steps in a software program. Such software may be employed in, for example, a digital signal processor, micro-controller, or general-purpose computer.

The present invention can be embodied in the form of methods and apparatuses for practicing those methods. The present invention can also be embodied in the form of program code embodied in tangible media, such as magnetic recording media, optical recording media, solid state memory, floppy diskettes, CD-ROMs, hard drives, or any other non-transitory machine-readable storage medium, wherein, when the program code is loaded into and executed by a machine, such as a computer, the machine becomes an apparatus for practicing the invention. The present invention can also be embodied in the form of program code, for example, stored in a non-transitory machine-readable storage medium including being loaded into and/or executed by a machine, wherein, when the program code is loaded into and executed by a machine, such as a computer, the machine becomes an apparatus for practicing the invention. When implemented on a general-purpose processor, the program code segments combine with the processor to provide a unique device that operates analogously to specific logic circuits.

It will be further understood that various changes in the details, materials, and arrangements of the parts which have been described and illustrated in order to explain the nature of this invention may be made by those skilled in the art without departing from the scope of the invention as expressed in the following claims.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

Unless explicitly stated otherwise, each numerical value and range should be interpreted as being approximate as if the word “about” or “approximately” preceded the value of the value or range. As used in this application, unless otherwise explicitly indicated, the term “connected” is intended to cover both direct and indirect connections between elements.

For purposes of this description, the terms “couple,” “coupling,” “coupled,” “connect,” “connecting,” or “connected” refer to any manner known in the art or later developed in which energy is allowed to be transferred between two or more elements, and the interposition of one or more additional elements is contemplated, although not required. The terms “directly coupled,” “directly connected,” etc., imply that the connected elements are either contiguous or connected via a conductor for the transferred energy.

The use of figure numbers and/or figure reference labels in the claims is intended to identify one or more possible embodiments of the claimed subject matter in order to facilitate the interpretation of the claims. Such use is not to be construed as limiting the scope of those claims to the embodiments shown in the corresponding figures.

The embodiments covered by the claims in this application are limited to embodiments that (1) are enabled by this specification and (2) correspond to statutory subject matter. Non-enabled embodiments and embodiments that correspond to non-statutory subject matter are explicitly disclaimed even if they fall within the scope of the claims.

Although the steps in the following method claims are recited in a particular sequence with corresponding labeling, unless the claim recitations otherwise imply a particular sequence for implementing some or all of those steps, those steps are not necessarily intended to be limited to being implemented in that particular sequence.

We claim:

1. An anesthesia-awareness detection (AAD) system comprising:
an output module adapted to be activated to generate at least one of an audible output and a visible output;
a muscle-motion sensor adapted to (i) detect one or more muscle-motion signals produced by an anesthetized patient and (ii) generate a sensor output signal corresponding to the one or more muscle-motion signals; and
a signal processor adapted to (i) process the sensor output signal to determine whether or not the one or more muscle-motion signals are volitional by the patient and (ii) activate the output module if the signal processor determines that the one or more muscle-motion signals are volitional indicating that the patient is aware.

2. The system of claim 1, wherein the muscle-motion sensor comprises a set of one or more surface electrodes adapted to:
be placed over a muscle of the patient;
detect electromyography (EMG) signals generated by the muscle; and
generate the sensor output signal to indicate the detected EMG signals.

3. The system of claim 2, wherein the set of surface electrodes comprises two or more surface electrodes arranged as an n-by-m grid.

4. The system of claim 2, wherein the set of surface electrodes is further adapted to:
be placed on a patient's forehead;
detect electroencephalogram (EEG) signals; and
generate an EEG output signal to indicate the detected EEG signals, wherein the EEG output signal is not the sensor output signal.

5. The system of claim 1, wherein the muscle-motion sensor comprises one or more accelerometers adapted to:
be placed over a muscle of the patient;
detect movements of the muscle; and
generate the sensor output signal to indicate the detected movements of the muscle.

6. The system of claim 1, wherein the muscle-motion sensor comprises a detector adapted to:
be placed proximate to a motor neuron associated with a muscle;
detect a neuromuscular (NM) signal generated by at least one of (i) the release of neurotransmitters by the motor neuron and (ii) the transmission of an impulse along the motor neuron; and
generate the sensor output signal indicative of the detected NM signal.

7. The system of claim 1, wherein the one or more muscle-motion signals include at least two of (i) an electromyography (EMG) signal, (ii) a neuromuscular (NM) signal, and (iii) an accelerometer-detectable muscle-motion signal.

8. The system of claim 1, wherein the muscle-motion sensor comprises a plurality of probe electrodes adapted to:
be inserted into a muscle of the patient;
detect electromyography (EMG) signals generated by the muscle; and
generate the sensor output signal to indicate the detected EMG signals.

9. The system of claim 1, wherein the signal processor is adapted to:
calculate a metric based on the sensor output signal; and
determine that the patient is aware if the metric exceeds a threshold that was set during a pre-operative preparation of the patient.

10. The system of claim 1, further comprising an automated consciousness-monitoring system adapted to determine and indicate that the patient is one of (i) aware and (ii) likely to soon become aware, wherein the determination is not based on muscle-motion signals produced by the patient.

11. The system of claim 1, wherein the signal processor is adapted to:
detect a plurality of particular patterns in the sensor output signal; and
activate the output module differently for each different detected particular pattern.

12. the system of claim 11, wherein the processor is programmable to define the plurality of particular patterns and the corresponding different output-module activations.

13. A method for detecting anesthesia awareness comprising:
detecting one or more muscle-motion signals produced by an anesthetized patient;
generating a sensor output signal corresponding to the one or more muscle-motion signals;
processing the sensor output signal to determine whether or not the one or more muscle-motion signals are volitional by the patient is aware; and
activating an output module if the signal processor determines that the one ore more muscle-motion signals are volitional indicating that the patient is aware, wherein the output module generates at least one of an audible output and a visible output if activated.

14. The method of claim 13, wherein:
the method further comprises placing a plurality of surface electrodes over a muscle of the patient, the surface electrodes adapted to detect electromyography (EMG) signals;
the detecting comprises detecting EMG signals generated by the muscle; and
the sensor output signal indicates the detected EMG signals.

15. The method of claim 13, wherein:
the method further comprises placing one or more accelerometers over a muscle of the patient adapted to detect movements of the muscle;
the detecting comprises detecting accelerometer-detectable movements of the muscle; and
the sensor output signal indicates the detected movements of the muscle.

16. The method of claim 13, wherein:
the method further comprises placing one or more muscle-motion sensor proximate to a motor neuron associated with a muscle, the sensor adapted to detect neuromuscular (NM) signals;
the detecting comprises detecting NM signals generated by at least one of (i) the release of neurotransmitters by the motor neuron and (ii) the transmission of an impulse along the motor neuron; and
the sensor output signal indicates the detected NM signal.

17. The method of claim 13, wherein the processing further comprises:
calculating a metric based on the sensor output signal; and
determining that the patient is aware if the metric exceeds a threshold that was set during a pre-operative preparation of the patient.

18. The method of claim 13, wherein the processing further comprises:
detecting a plurality of particular patterns in the sensor output signal; and
activating the output module differently for each different detected particular pattern.

19. The method of claim 18, further comprising programming a processor to define the plurality of particular patterns and the corresponding different output-module activations.

20. The method of claim 13, further comprising administering anesthetics to the patient such that the patient retains the ability to at-least-partially furrow his or her brow while otherwise substantially immobilized.

21. A method comprising:
anesthetizing a patient;
then determining to assess the consciousness of the anesthetized patient;
then asking the anesthetized patient to perform an activity that would generate a detectable muscle-motion signal;
then observing an output module of an anesthesia-awareness system to determine whether a corresponding muscle-motion signal is detected;
then determining that:
the patient is aware if the corresponding muscle-motion signal is detected; and
the patient is probably not aware if the corresponding muscle-motion signal is not detected; and
then administering additional sedative to the patient if it was determined that the patient is aware.
